(19) United States
(12) Patent Application Publication
(10) Pub. No.: US 2007/0015187 A1
(43) Pub. Date: Jan. 18, 2007

(54) METHODS FOR RNA PROFILING

(75) Inventors: Kai Qin Lao, Pleasanton, CA (US); Kenneth J. Livak, San Jose, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/458,086

(22) Filed: Jul. 17, 2006

Related U.S. Application Data
(60) Provisional application No. 60/699,953, filed on Jul. 15, 2005.
(60) Provisional application No. 60/710,380, filed on Aug. 23, 2005.

(51) Int. Cl. C12Q 1/68; C12P 19/34

(52) U.S. Cl. 435/6; 435/91.2

(57) ABSTRACT

The present teachings provide methods, compositions, and kits for detecting micro RNAs (miRNAs). In some embodiments, the miRNAs are quantified from formalin-fixed paraffin-embedded tissue samples in which messenger RNA is degraded. The present teachings take advantage of the observation that most mature miRNAs in vivo are protected by degradation as a result of their association with RISC. Thus, novel methods of studying nucleic acids in archived tissues containing degraded messenger RNA are provided, wherein RISC-protected miRNAs are liberated, and analyzed.

### CROSS-REFERENCE TO RELATED APPLICATIONS

This application claims a benefit of priority under 35 U.S.C. §119(e) from U.S. Patent Application No. 60/699,953, filed Jul. 15, 2005 and U.S. Patent Application No. 60/710,380, filed Aug. 23, 2005, the contents of each are incorporated herein by reference.

### FIELD

The present teachings relate to methods of measuring and profiling micro RNAs (miRNAs), especially in tissues that contain degraded messenger RNAs.

### INTRODUCTION

Analysis of expressed nucleic acids can be difficult in small and limited samples. Approaches that multiplex nucleic acid analyses are of growing importance in the biomedical research community. Micro RNAs (miRNAs) are increasingly recognized class of nucleic acids that play important roles in human disease, including cancers (see Lu et al., 2005, Nature 435: 834-838).

There is a vast supply of formalin-fixed, paraffin-embedded tumor tissues for which response to treatment and clinical outcome is already known. Although these archived tissues can be used for in situ techniques to show localization of gene expression (see for example Steele et al., Cell Vis 1998, 5:13-19, and Gruber et al., J Virol Methods 1993, 43:309-319) the RNA is often too degraded for classical, quantitative analysis methods such as Northern blots. Reverse transcription-polymerase chain reaction (RT-PCR) has been used extensively to detect expression of genes in cultured cells and in fresh or frozen tissues, and recent technological advances now allow rapid and accurate quantitative RT-PCR analyses (Lie et al., Curr Opin Biotechnol 1998, 9:43-48, and Heid et al., Genome Res 1996, 6:986-994). Furthermore, the ability of RT-PCR to assay very small fragments of mRNAs makes this technique amenable to studies where the RNA is moderately or even highly degraded, as in the case of RNA from archived tissues (see for example Inoue et al., Pathol Int 1996, 46:997-1004, and Tyrrell et al., Am J Dermatopathol 1995, 17:476-483). Despite considerable progress in sample preparation (see Godfrey et al., Journal of Molecular Diagnostics, 2000, 2:2, 84-91, and Lewis and Maughan in Bustin, A-Z of Quantitative PCR, 2004), profiling of nucleic acids in archived tissues remains problematic.

The detection of very small fragments of nucleic acids (e.g. miRNAs) can be difficult with conventional PCR, due to for example overlapping primers on the short target producing primer dimer artifacts. Recently, techniques that take advantage of the sensitivity, specificity, and dynamic range of quantitative real-time PCR have been invented that allow for the quantitation of such short nucleic acids (see for example U.S. Non-Provisional application Ser. No. 10/881,362 to Brandis et al., Ser. No. 10/944,153 to Lao et al., Ser. No. 10/947,460 to Chen et al., and Ser. No. 11/142,720 to Chen et al.,)

### SUMMARY

In some embodiments, the present teachings provide a method of detecting non-degraded mature miRNA in a sample, wherein the sample comprises substantially degraded messenger RNA, said method comprising; liberating the mature miRNA to form a collection of pure mature miRNAs; with the proviso that the liberating does not comprise treating with a detergent and does not comprise treating with a chaotropic salt; forming a reaction mixture comprising the non-degraded pure mature miRNAs, and at least one miRNA specific reverse primer; extending the miRNA specific reverse primer to form a miRNA extension product; amplifiying the miRNA extension product; and, detecting the non-degraded mature miRNA in the sample. In some embodiments, additional methods, as well as kits are also provided. For example, in some embodiments the present teachings provide a kit for detecting non-degraded mature miRNA in a sample wherein the sample comprises substantially degraded messenger RNA, said kit comprising; (a) reagents for extracting nucleic acids from a tissue section containing substantially degraded messenger RNA; and, (b) an RNAse.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a forward primer” means that more than one forward primer can be present; for example, one or more copies of a particular forward primer species, as well as one or more different forward primer species. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Some Definitions

As used herein, the term “substantially degraded mRNA” refers to a state in which a significant percentage of the mRNA is degraded. Measures of degradation of mRNA are well known to one of ordinary skill in the art of molecular biology and include measuring the ratio of 28S to 18S rRNA on membranes derived from agarose gels, the commercially available Agilent 2100 bioanalyzer, and other approaches as discussed for example in Sambrook et al., Molecular Cloning 3rd Edition. In some embodiments, substantially degraded mRNA can comprise more than 90 percent degradation relative to a matched un-degraded sample. In some embodiments, substantially degraded mRNA can comprise between 80-90 percent degradation relative to a matched un-degraded sample. In some embodiments, substantially degraded mRNA can comprise between 70-80 percent degradation relative to a matched un-degraded sample. In some embodiments, substantially degraded mRNA can comprise between 60-70 percent degradation relative to a matched un-degraded sample. In some embodiments, substantially degraded mRNA can comprise between 50-60 percent degradation relative to a matched un-degraded sample. In some embodiments, substantially degraded mRNA can comprise 10-50 percent degradation relative to a matched un-degraded sample.

As used herein, the term “RISC-protected mature miRNA” refers to a collection of miRNA species that are complexed with a RISC molecule, and hence resistant to degradation. These RISC-protected mature miRNAs have already undergone processing, and are not pri-miRNAs or pre-miRNAs. Without being limited to any particular theory, RISC-protected mature miRNAs can be bound directly to RISC, bound to RISC through an intermediary(s), or both.

As used here, the term “liberating the mature miRNA” refers to a process whereby mature miRNAs are released from the RISC complex, thus forming a plurality of free, single-stranded mature miRNAs. The process of liberating can comprise any of a variety of methods known to release nucleic acids from proteins. For example, liberating can comprise applying heat, for example 95 C for 5 minutes. In some embodiments, liberating can comprise applying heat, for example 80 C or higher for 5 minutes. Of course, routine experimentation can yield other times and temperatures suitable for heat-based liberating of mature miRNAs. Liberating can comprise treatment with a detergent, for example 10% SDS. Liberating can also comprise treatment with a chaotropic salt, such as for example guanidinium-based compounds.

As used herein, the term “additional nucleic acids” refers to a collection of nucleic acids that are not mature miRNAs. Included in the term additional nucleic acids are molecules such as pri-miRNAs and pre-miRNAs, as well as other non-coding RNAs, messenger RNAs, transfer RNAs, ribosomal RNAs, and genomic DNA.

As used herein, the term “pure mature miRNAs” refers to a collection of mature miRNAs that are free of additional nucleic acids, are no longer associated with RISC, and are 18-23 nucleotides in length.

As used herein, the term “experimentally-added active nuclease” refers to a nuclease, such as for example an RNAse and/or a DNAse, which is not present endogenously in a sample, but rather is added by an experimentalist. The nuclease is active, in that it can degrade, for example, additional nucleic acids.

As used herein, the term “experimentally-added nuclease that is inactivated” refers to a nuclease, such as for example an RNAse and/or a DNAse, which is not present endogenously in a sample, but rather is added by an experimentalist. The nuclease is originally active, in that it can degrade, for example, additional nucleic acids. The nuclease is later inactivated, for example by treatment with heat and/or a protease, thus resulting in an experimentally-added nuclease that is inactivated.

As used herein, the term “heating for a sufficient time and a sufficient temperature to lyse cells and free the additional nucleic acids without liberating mature miRNA” refers to an empirically determined set of time and temperature conditions for a given sample, easily derived by one of skill the art of molecular biology. Such conditions can be measured, by for example, performing a PCR on a target nucleic acid (a messenger RNA) that is desired to be liberated, and ensuring the presence of that target nucleic acid in the lysate, and the absence of that target nucleic in the sample before lysis. Correspondingly, the absence of a free mature miRNA in the lysate, as well as in the unlysed sample, can be determined using an amplification-based assessment. In some embodiments, heating for a sufficient time and a sufficient temperature to lyse cells and free the additional nucleic acids without liberating mature miRNA shall mean that at least 50 percent of the additional nucleic acids are free relative to a non-lysed sample, and that less than 25 percent of mature miRNAs are free relative to a non-lysed sample. In some embodiments, heating for a sufficient time and a sufficient temperature to lyse cells and free the additional nucleic acids without liberating mature miRNA shall mean that at least 75 percent of the additional nucleic acids are free relative to a non-lysed sample, and that less than 10 percent of mature miRNAs are free relative to a non-lysed sample. In some embodiments, heating for a sufficient time and a sufficient temperature to lyse cells and free the additional nucleic acids without liberating mature miRNA shall mean that at least 90 percent of the additional nucleic acids are free relative to a non-lysed sample, and that less than 5 percent of mature miRNAs are free relative to a non-lysed sample. In some embodiments, heating for a sufficient time and a sufficient temperature to lyse cells and free the additional nucleic acids without liberating mature miRNA shall mean that at least 99 percent of the additional nucleic acids are free relative to a non-lysed sample, and that less than 1 percent of mature miRNAs are free relative to a non-lysed sample.

As used herein, the term “detector probe” refers to a molecule used in an amplification reaction, typically for quantitative or real-time PCR analysis, as well as end-point analysis. Such detector probes can be used to monitor the amplification of the target miRNA and/or control nucleic acids such as endogenous control small nucleic acids and/or synthetic internal controls. In some embodiments, detector probes present in an amplification reaction are suitable for monitoring the amount of amplicon(s) produced as a function of time. Such detector probes include, but are not limited to, the 5′-exonuclease assay (TaqMan® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Illustrative detector probes comprising two probes wherein one molecule is an L-DNA and the other molecule is a PNA can be found in U.S. Non-Provisional patent application Ser. No. 11/172,280 to Lao et al., Detector probes can also comprise sulfonate derivatives of fluorescenin dyes with SO3 instead of the carboxylate group, phosphoramidite forms of fluorescein, phosphoramidite forms of CY 5 (commercially available for example from Amersham). In some embodiments, intercalating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, probes can further comprise various modifications such as a minor groove binder (see for example U.S. Pat. No. 6,486,308) to further provide desirable thermodynamic characteristics. In some embodiments, detector probes can correspond to identifying portions or identifying portion complements, also referred to as zip-codes. Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein).

The primers and probes of the present teachings can contain conventional nucleotides, as well as any of a variety of analogs. For example, the term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures: 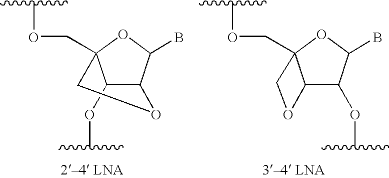
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

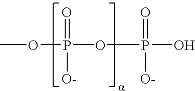

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

Other terms as used herein will harbor meaning based on the context, and can be further understood in light of the understanding of one of skill in the art of molecular biology. Illustrative teachings describing the state of the art can be found, for example, in Sambrook et al., Molecular Cloning, 3rd Edition.

We have shown that in cells under physiological conditions, most miRNAs are tightly associated with RISC complexes and only a small fraction and possibly less than three percent are present outside RISCs. See co-filed U.S. Non-Provisional Application Pure miRNA Sample Preparation Method to Lao et al., claiming priority to U.S. Provisional Application 60/780,927. To our knowledge, this is the first time that endogenous miRNAs have been demonstrated to be tightly associated with RISCs in vivo under physiological conditions. These observations may explain why miRNAs are so stable in cells, and it also explains why they exhibit a very long half-life. Further, these observations provide the basis for the present teachings. In various tissue samples, messenger RNAs are degraded. There is a desire to study such samples. The present teachings provide for methods of analyzing samples in which the messenger RNA is degraded, by taking advantage of the stability of miRNAs by virtue of the association with RISC in vivo.

Description of Experiments Illustrating that Most Mature miRNAs in Cells are RISC-Protected

We employed stem-loop reverse transcription real-time PCR assays (see U.S. patent application Ser. No. 10/947,460 and Chen et al., Nucleic Acids Res. Nov. 27, 2005;33(20):e179) to detect endogenous miRNAs in a cell. In short, a reverse primer comprising a single-stranded target-specific region complementary to a given miRNA is employed. The reverse primer also contains a double stranded stem, and a single stranded loop. This stem-loop primer is extended in a reverse transcription reaction. Thereafter, a PCR is performed. The reverse primer in the PCR was encoded in the loop of the stem-loop primer, and the forward primer in the PCR comprises a target-specific region, and a non-complementary tail. The accumulation of reaction products in the PCR is measured using a 5′ nuclease detector probe.

These studies verified that this assay is specific to mature miRNAs, and it can easily discriminate between mature miRNA, genomic DNA from which miRNAs originate, primary miRNAs (Pri-miRNAs), and precursor miRNAs (Pre-miRNAs). Furthermore, the assay is very sensitive because it can detect miRNA in pg amounts of total RNA sample. This approach therefore offers a unique opportunity to detect endogenous levels of miRNAs directly and without modifications. As a result of employing this real-time PCR based method to find out what proportion of miRNAs are associated with RISCs in vivo in a cell under physiological conditions, the present teachings provide novel methods, compositions, and kits for studying miRNAs.

First, we established conditions that release all miRNAs from cells. After a series of tests, we found that treatment of cells at 95 C for 5 minutes can release most if not all miRNAs from ES cells. MirVana is a well-known reagent that is highly efficient for the isolation of small RNAs, including miRNAs, from cells or tissues. Treatment at 95 C for 5 minutes and MirVana showed similar efficiency for the release of miRNAs from cells.

Second, we sought to establish a milder condition that can completely release RNA from cells, but without destroying stable protein complexes. From our investigations, we found that subjecting cells to three freeze-thaw cycles lysed ES cells, as confirmed by microscopic examination that showed that most cells were lysed after this treatment. We then checked for the release of mRNA of a housekeeping gene, GAPDH that is highly expressed in cells, and found that most of the GAPDH mRNA was released from ES cells after freeze-thawing. Further treatment of the sample at 95 C for 5 minutes did not release any further mRNA. To confirm that this was the case, we also checked for the release of 18s rRNA following this treatment, which confirmed the release of most of the 18s rRNA from ES cells. These data show that three freeze-thaw cycles can release most of the free RNAs present in cells.

Next, we examined the levels of endogenous miRNA that are associated with RISC complexes in cells by stem-loop RT-PCR. MiR-16 and mir-20 are two highly expressed miRNAs in ES cells. When we compared the levels of miR-16 released following treatment at 95 C for 5 minutes with the levels detected following three freeze-thaw cycles, we found at least 30-fold more miRNA following the first treatment. Thus it seems that the levels of free miR-16 in ES cells are less than 3% of the total miR-1 6 present in ES cells. In other words, under physiological conditions, most of the miR-16 is stably associated with RISC complexes. We similarly checked the levels of miR-16 in another cell line, NIH/3T3 cells, and in primary mouse embryonic fibroblasts (MEFs). We found that the levels of free miR-16 accounted for at most only 1-2 percent of all miR-16 present in NIH/3T3 cells and MEFs. To exclude any sequence specific bias in our analysis, we also examined the levels of miR-20, another highly expressed miRNA, and again found a similar ratio of free versus bound miRNA. Therefore, we conclude that under physiological conditions in vivo, most of the miRNAs are tightly associated with RISC complexes, and possibly only 1-3 percent, if any, are free in cells.

To confirm that the miRNAs released by freeze-thaw cycles are indeed free miRNA, we treated the samples with RNase I. RNase I is known to degrade any RNAs into a mixture of mono-, di-, and trinucleotides. First, we confirmed that RNase I treatment at 37 C for 5 minutes can indeed completely degrade free miRNAs. RNase I treatment of miRNA obtained following treatment of cells at 95 C for 5 minutes, did indeed degrade all miRNAs to background levels. Second, we found that the samples obtained after three freeze-thaw cycles when treated with RNase I for 5 minutes, did not degrade miRNAs associated with RISC dramatically. Finally, we compared cell lysate containing intact RISCs (following three freeze-thaw cycles) to samples that were treated at 95 C for 5 minutes that destroys the RISC complex. RNase I treatment of these samples showed that less than one percent miRNAs were degraded in samples following the first treatment compared to the latter samples. This demonstrates that nearly all miRNAs are tightly associated with RISC complexes in cells and only a tiny fraction of miRNA are free in intact cells under physiological conditions.

We next considered a possible argument that we may be underestimating free miRNA in cells because any endogenous RNases in cell lysate (but not in living cells) may degrade free miRNAs dramatically. To exclude this possibility, we added purified total RNA (including mature miRNAs) into cell lysate obtained after three freeze-thaw cycles. Our analysis showed that incubating mature miRNA in cell lysate did not cause their degradation to any significant degree. This provides evidence that the levels of miRNA in cell lysate are not underestimated in our assay.

Next, we set out to determine the stability of the miRNA association with the RISC complex. To test this, we treated ES cells at different temperatures and found that miR-16 was stably associated with the RISC complex even after treatment at 60 C for 5 minutes. It was only following treatment of the samples at 80 C for 5 minutes that most of the miR-16s were released from the RISC complexes. These observations suggest that most of the endogenous miRNAs are tightly associated with RISC complexes in cells.

It has previously been reported that siRNA can bind stably to RISC complexes even in the presence of 2.5M NaCl, 2.5M KCl, or 1M Urea. We found that the endogenous miR-16 was stably associated with RISC even following treatment with 2.5M NaCl or KCl or ES cell lysates. Treating ES cell lysates obtained following three freeze-thaw cycles with 2.5M NaCl or KCl did not show a dramatic increase in the release of miRNA. These data suggest that endogenous miRNAs are tightly bound to RISCs in cells, which resembles the stable association between siRNA/RISC in vitro. Next, we wanted to assess the dynamics of the association between miRNAs and RISC complexes. To test this, we loaded 0.5 uM synthetic Pre-miRNA or mature miRNA into ES cell lysate and incubated the sample for 30 minutes. We then checked the amount of miR-16 associated with RISC in the cell lysate. We found that neither adding Pre-miRNA nor mature miRNA of Let-7a could release miR-16 from RISC complexes. Adding 0.5 uM synthetic single-stranded mature miRNAs could not replace endogenous miRNAs in RISC complexes, which indicates that the relative abundance of miRNAs is determined by the amount of initial primary miRNA transcripts. Adding 0.5 uM synthetic hairpin precursor Let-7a (Pre-Let-7a) also could not replace endogenous miRNAs from their RISC complexes. Without intending to limited by any particular theory, these data suggest the following possibilities. First, the replacement kinetic may be very slow and it may take hours or more to see an effect. Second, precursors may be “transported” into RISC complexes by other protein complexes in living cells. This suggests that the miRNA/RISC association is very stable and free miRNA cannot significantly replace miRNA that is already present in RISC complexes.

Recently, a novel chemically modified oligonucleotide, referred to as ‘antagomirs,’ have been shown to be able to knockdown complementary miRNA by promoting their degradation (Krutzfeldt et al., Nature, 438, 685-689). We proposed that one possibility for this mechanism is that antagomir molecules may bind to target miRNA within RISC and dissociate it from this complex. RNases may subsequently degrade dissociated miRNA/antagomir hybrid (the miRNA strand) released in the cell cytoplasm. To test this possibility, we incubated the ES cell lysate obtained following three freeze-thaw cycles described above, with 0.5 nM-50 nM of antagomirs of miR-16 (antagomir-16) for 30 minutes. The sample was then treated with RNAse I to degrade miR-16 that may have been released from RISC complexes. Finally, RNase I was inactivated and the remaining RISC-bound miRNAs were released by treatment of the ES cell lysate at 95 C for 5 minutes, and their levels were estimated by RT-PCR. We found that incubation of the sample with 5 nM of antagomir-16 can degrade 90% of miR-16 in ES cell lysate (the antagomir-16 effect of blocking stem-loop RT-PCR reaction have been corrected by comparing to the same amount of synthetic miR-16 for 0.5-50 nM antagomirs data). As a control for the antagomirs, we used antisense miR-16 RNA molecule, or an unrelated antagomir-let-7a, but neither of them were able to promote the release and subsequent degradation of miR-16 from RISC in the freeze-thaw ES cell lysate. We also checked the antagomir effect in a similar lysate from MEFs, and obtained similar results. Without being limited by any particular theory, the data are thus compatible with the notion that antagomirs promote dissociation of specific miRNAs from RISC, and this is followed by their subsequent degradation.

Finally, we verified that the stem-loop RT-PCR is specific to mature miRNA and can discriminate between mature miRNAs from their precursors unequivocally. For this purpose, we measured miR-16 expression in MEFs that lack Dicer. It is known that in such Dicer null cells, mature miRNAs are absent and there is an accumulation of pri-miRNAs and pre-miRNAs (Kanellopoulou et al., Genes Dev., 19, 489-501, and Murchison et al., Proc. Natl. Acad. Sci. USA, 102, 12135-12140). We indeed found that compared to wild-type MEFs, there is virtually no detectable miR-16. These data provide additional evidence that stem-loop RT-PCR does specifically detect mature miRNA, and not the corresponding genomic locus, pre-miRNA, or pre-miRNA.

In general, these data show that in cells under physiological conditions, most miRNAs are tightly associated with RISC complexes and only a small fraction and possibly less than three percent are present outside RISCs. To our knowledge, this is the first time that endogenous miRNAs have been demonstrated to be tightly associated with RISCs in vivo under physiological conditions. These observations may explain why miRNAs are so stable in cells, and it also explains why they exhibit a very long half-life. Our study also shows that antagomirs can effectively and specifically displace miRNA from RISC, which is followed by their degradation.

Some Applications of the Present Teachings

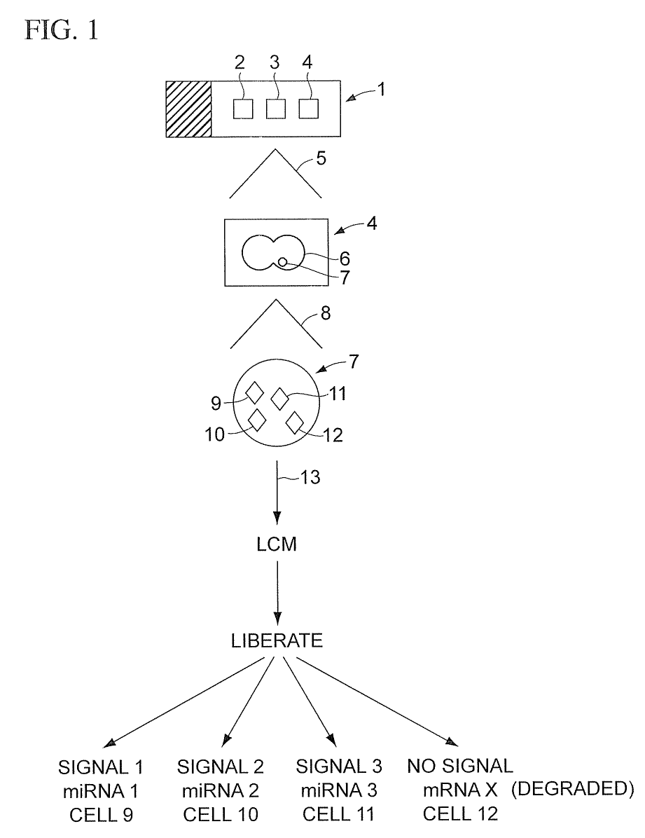

FIG. 1 depicts one work-flow according to some embodiments of the present teachings. Here, a microslide slide (1) is shown containing three tissue samples (2, 3, 4), for example formalin-fixed paraffin-embedded tissue slices. (For this illustration, the tissue samples are archived, and old, such that the messenger RNA is degraded.) One of these tissue samples (4) is shown magnified, wherein a coronal brain section is shown (6), containing a nucleus of cells (7), perhaps the central nucleus of the amygdala. Further, this nucleus of cells (7) is shown magnified, wherein four different cells reside (9, 10, 11, 12). Laser Capture Microscopy (LCM, as is commercially available from Arcturus) can be employed to isolate each of the four cells (9, 10, 11, and 12). Each of the four cells can be placed in a reaction vessel. (At this point, if the tissue section is a formalin-fixed paraffin-embedded section, the sample can be deparaffinized by incubation with xylene and with 100% ethanol, and subsequent drying of the resulting tissue pellet. Such procedures for deparaffiizing are routine, and can be found described for example in Steg et al., Journal of Molecular Diagnostics, Vol. 8, No. 1, February 2006, as well as the January 1006 version of the manual for the High Pure RNA Paraffin Kit, commercially available from Roche Applied Science, and the RecoverAll TM kit commercially available from Ambion). Next, the non-degraded mature miRNAs can be liberated, for example by heating for 5 minutes at 95 C. Thereafter, each of the four reaction vessels can undergo a PCR amplification, using for example a target-specific stem-loop RT primer. The results of the amplification can produce signal, either in real-time or as an end-point. In those cells (9, 10, 11) in which miRNAs were reverse-transcripted and PCR amplified with miRNA specific primers, signal will result, thus allowing for quantitation of a particular miRNA. However, in the reaction vessel corresponding to cell 12, primers querying a messenger RNA are employed. No signal results due to the degradation of the messenger in the original tissue section. (Of course, any non-degraded messenger RNA in the original tissue section would likely be destroyed by the liberating of the mature miRNA.)

Thus, in some embodiments the present teachings provide method of detecting non-degraded mature miRNA (miRNA) in a sample, wherein the sample comprises substantially degraded messenger RNA (mRNA), said method comprising; liberating the mature miRNA to form a collection of pure mature miRNAs; with the proviso that the liberating does not comprise treating with a detergent and does not comprise treating with a chaotropic salt; forming a reaction mixture comprising the non-degraded pure mature miRNAs, and at least one miRNA specific reverse primer; extending the miRNA specific reverse primer to form a miRNA extension product; amplifiying the miRNA extension product; and, detecting the non-degraded mature miRNA in the sample

In some embodiments, the sample is a formalin-fixed paraffin embedded tissue section. In some embodiments, the detecting comprises a polymerase chain reaction (PCR). In some embodiments, the liberating comprises heating. In some embodiments, the heating comprises at least 70 C for 5 minutes. In some embodiments, the heating comprises 95 C for 5 minutes.

In some embodiments, the present teachings provide a method of detecting non-degraded mature miRNA (miRNA) in a sample, wherein the sample comprises substantially degraded messenger RNA (mRNA), said method comprising; lysing the sample; treating the sample with a nuclease; inactivating the nuclease; liberating the non-degraded mature miRNAs from the RISC complex; forming a reaction mixture comprising the non-degraded pure mature miRNAs, and at least one miRNA specific reverse primer; extending the miRNA specific reverse primer to form a miRNA extension product; amplifiying the miRNA extension product; and, detecting the non-degraded mature miRNA in the sample. In some embodiments, the liberating comprises treating with a detergent. In some embodiments, the liberating comprises heating. In some embodiments, the heating comprises at least 70 C for 5 minutes. In some embodiments, the heating comprises 95 C for 5 minutes.

In some embodiments, the present teachings provide a method of detecting non-degraded mature miRNA (miRNA) in a sample wherein the sample comprises substantially degraded messenger RNA (mRNA), said method comprising; liberating the mature miRNA to form a collection of pure mature miRNAs; forming a reaction mixture comprising the non-degraded pure mature miRNAs, and at least one miRNA specific reverse primer; extending the miRNA specific reverse primer to form a miRNA extension product; amplifying the miRNA extension product in a PCR; and, detecting the mature miRNA in the sample, wherein the sample is a formalin-fixed paraffin embedded tissue section. In some embodiments, the detecting comprises a polymerase chain reaction (PCR). In some embodiments, the liberating comprises heating. In some embodiments, the heating comprises at least 70 C for 5 minutes. In some embodiments, the heating comprises 95 C for 5 minutes.

In some embodiments, the degrading comprises treatment with at least one nuclease. In some embodiments, the at least one nuclease is an RNAse. An RNAse can be helpful, for example, in the degradation of precursor micro RNAs not associated with RISC, as well as other RNAs in the cell lysate, such as for example messenger RNAs, transfer RNAs, ribosomal RNAs, and various non-coding RNAs. In some embodiments,the RNAse is RNAse I. In some embodiments, the at least one nuclease is a DNAse. A DNAse can be helpful, for example, in the degradation of genomic DNA present in the cell lysate. In some embodiments, the DNAse is DNAse I. In some embodiments, the at least one nuclease comprises an RNAse and a DNAse. Of course, any of a variety of nuclease are commercially available and can be used in the present teachings, for example as can be purchased from New England Biolabs. Further descriptions of various nuclease, and their used in degrading unwanted nucleic acids, can be found, for example in U.S. patent application Ser. No. 10/982,619, and U.S. Pat. No. 6,797,470.

In some embodiments, the methods of the present teachings provide for the quantitation of miRNAs from tissue samples comprising degraded messenger RNA that are formalin-fixed paraffin embedded tissue samples, wherein the quantitation provides nearly the same levels of measured miRNAs as are found, or would be found, in experimentally matched tissue samples that do not comprise degraded messenger RNA and/or are formalin-fixed paraffin embedded tissue samples (e.g.-fresh frozen tissue samples).

Various reverse transcriptases can be used when reverse transcribing the miRNAs obtained by the present teachings, as are readily available to the molecular biology experimentalist, including for example MMLV and rTth, and various commercially available reverse transcriptases available from New England Biolabs, Applied Biosystems, Ambion, and Stratagene.

In some embodiments, the sensitivity of detection is one molecule of a target miRNA. In some embodiments, the sensitivity of detection is five or fewer molecules of target miRNA. In some embodiments, the sensitivity of detection is twenty-five or fewer molecules of target miRNA. In some embodiments, the sensitivity of detection is fifty or fewer molecules of target miRNA. In some embodiments, the sensitivity of detection is one hundred or fewer molecules of target miRNA. In some embodiments, the sensitivity of detection is one thousand or fewer molecules of target miRNA.

In some embodiments, the dynamic range of at least two signals from quantified miRNAs is at least three log units. In some embodiments, the dynamic range of at least two signals from quantified miRNAs is at least four log units. In some embodiments, the dynamic range of at least two signals from quantified miRNAs is at least four log units. In some embodiments, the dynamic range of at least two signals from quantified miRNAs is at least five log units. In some embodiments, the dynamic range of at least two signals from quantified miRNAs is at least six log units. In some embodiments, the dynamic range of at least two signals from quantified miRNAs is greater than six log units.

Additional description of the stability of miRNAs can be found in co-filed U.S. Patent Application to Lao, claiming priority to U.S. Provisional 60/716,633 and U.S. Provisional Application 60/780,927. Armed with the present teachings, one of ordinary skill in the art can employ routine experimentation to further refine the duration and temperature ranges for preparing miRNAs for their amplification. Additional teachings for preparation of nucleic acids, in particular messenger RNA, can be found in (Godfrey et al., Journal of Molecular Diagnostics, 2000, 2:2, 84-91).

Certain methods of optimizing reverse transcription and amplification reactions are known to those skilled in the art. For example, it is known that PCR may be optimized by altering times and temperatures for annealing, polymerization, and denaturing, as well as changing the buffers, salts, and other reagents in the reaction composition. Optimization may also be affected by the design of the amplification primers used. For example, the length of the primers, as well as the G-C:A-T ratio may alter the efficiency of primer annealing, thus altering the amplification reaction. Descriptions of amplification optimization can be found in, among other places, James G. Wetmur, “Nucleic Acid Hybrids, Formation and Structure,” in Molecular Biology and Biotechnology, pp. 605-8, (Robert A. Meyers ed., 1995); McPherson, particularly in Chapter 4; Rapley; and Protocols & Applications Guide, rev. 9/04, Promega.

In some embodiments, the present teachings can be applied in various contexts with co-filed U.S. Non-Provisional Application “Hot Start Reverse Transcription by Primer Design” to Lao et al., claiming priority to 60/699,967, and co-filed U.S. Provisional Application “Analyzing Messenger RNA and MiRNA in the Same Reaction Mixture” to Lao et al., claiming priority to 60/699,930.

In some embodiments, the present teachings contemplate single-tube RT-PCR approaches, and discussed for example in Mohamed et al., (2004) Journal of Clinical Virology, 30:150-156.

In some embodiments, the present teachings contemplate various cycling reverse transcription reaction approaches, as discussed for example in U.S. Non-Provisional Application to Lao et al., Ser. No. 11/421,460, and U.S. Non-Provisional application Bloch et al., Ser. No. 11/421,319.

In some embodiments, the reverse transcription products of the present teachings can be amplified in a multiplexed pre-amplifying PCR followed by a plurality of lower-plex decoding PCRs, as described for example in WO2004/051218 to Andersen and Ruff, U.S. Pat. No. 6,605,451 to Xtrana, and U.S. Non-Provisional application Ser. No. 11/090,830 to Andersen et al., and U.S. Non-Provisional application Ser. No. 11,090,468 to Lao et al.,

In some embodiments, the methods of the present teachings can employ recently developed techniques that take advantage of the sensitivity, specificity, and dynamic range of quantitative real-time PCR for the quantitation miRNAs (see for example U.S. Non-Provisional application Ser. No. 10/881,362 to Brandis et al., Ser. No. 10/944,153 to Lao et al., Ser. No. 10/947,460 to Chen et al., and Ser. No. 11/142,720 to Chen et al.,). For example, in some embodiments, a miRNA specific “stem-loop” reverse primer is employed in a primer extension reaction followed by a real-time PCR, wherein the stem-loop primer comprises a self-complementary stem, a loop, and a single-stranded miRNA target specific region, as described for example in U.S. Non-Provisional patent application Ser. 10/947,460 to Chen et al., In some embodiments, the miRNAs collected by the present teachings can be further analyzed in highly multiplexed RT-PCR reactions, as taught for example Lao et al., U.S. patent application Ser. No. 11/421,449.

In some embodiments, miRNAs collected from tissue samples according to the present teachings can be analyzed on microarrays, for example LNA-based microarrays, as taught for example in Castoldi et al., May 2006;12(5):913-20. Epub Mar. 15, 2006.

Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

Thus, in some embodiments, the present teachings provide a kit for quantitating non-degraded mature miRNA (miRNA) in a sample wherein the sample comprises substantially degraded messenger RNA (mRNA), said kit comprising; (a) reagents for extracting nucleic acids from a tissue section containing substantially degraded messenger RNA; (b) an RNAse. In some embodiments, the reagents for extracting nucleic acids from the tissue section containing substantially degraded messenger RNA comprise xylene and ethanol, and the tissue section is paraffin-embedded. In some embodiments, the RNAse is RNAse I.

In some embodiments, the present teachings provide a kit for detecting non-degraded mature miRNA (miRNA) in a sample wherein the sample comprises substantially degraded messenger RNA (mRNA), said kit comprising; (a) reagents for extracting nucleic acids from a tissue section containing substantially degraded messenger RNA; and (b) an RNAse. In some embodiments, the reagents for extracting nucleic acids from the tissue section containing substantially degraded messenger RNA comprise xylene and ethanol, and the tissue section is paraffin-embedded. In some embodiments, the RNAse is RNAse I. In some embodiments, the kit comprises a DNAse.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

1. A method of detecting non-degraded mature miRNA in a sample, wherein the sample comprises substantially degraded messenger RNA, said method comprising;
liberating the mature miRNA to form a collection of pure mature miRNAs; with the proviso that the liberating does not comprise treating with a detergent and does not comprise treating with a chaotropic salt;
forming a reaction mixture comprising the non-degraded pure mature miRNAs, and at least one miRNA specific reverse primer;
extending the miRNA specific reverse primer to form a miRNA extension product;
amplifiying the miRNA extension product; and,
detecting the non-degraded mature miRNA in the sample.

2. The method according to claim 1 wherein the sample is a formalin-fixed paraffin embedded tissue section.

3. The method according to claim 1 wherein the detecting comprises a polymerase chain reaction (PCR).

4. The method according to claim 1 wherein the liberating comprises heating.

5. The method according to claim 4 wherein the heating comprises at least 70 C for 5 minutes.

6. The method according to claim 4 wherein the heating comprises 95 C for 5 minutes.

7. A method of detecting non-degraded mature miRNA in a sample, wherein the sample comprises substantially degraded messenger RNA, said method comprising;
lysing the sample;
treating the sample with a nuclease;
inactivating the nuclease;
liberating the non-degraded mature miRNAs from the RISC complex;
forming a reaction mixture comprising the non-degraded pure mature miRNAs, and at least one miRNA specific reverse primer;
extending the miRNA specific reverse primer to form a miRNA extension product;
amplifiying the miRNA extension product; and,
detecting the non-degraded mature miRNA in the sample.

8. The method according to claim 7 wherein the liberating comprises treating with a detergent.

9. The method according to claim 7 wherein the liberating comprises heating.

10. The method according to claim 9 wherein the heating comprises at least 70 C for 5 minutes.

11. The method according to claim 9 wherein the heating comprises 95 C for 5 minutes.

12. A method of detecting non-degraded mature miRNA in a sample wherein the sample comprises substantially degraded messenger RNA, said method comprising;
liberating the mature miRNA to form a collection of pure mature miRNAs;
forming a reaction mixture comprising the non-degraded pure mature miRNAs, and at least one miRNA specific reverse primer;
extending the miRNA specific reverse primer to form a miRNA extension product;
amplifying the miRNA extension product in a PCR; and,
detecting the mature miRNA in the sample, wherein the sample is a formalin-fixed paraffin embedded tissue section.

13. The method according to claim 12 wherein the detecting comprises a polymerase chain reaction (PCR).

14. The method according to claim 12 wherein the liberating comprises heating.

15. The method according to claim 14 wherein the heating comprises at least 70 C for 5 minutes.

16. The method according to claim 14 wherein the heating comprises 95 C for 5 minutes.

17. A kit for detecting non-degraded mature miRNA in a sample wherein the sample comprises substantially degraded messenger RNA, said kit comprising;
(a) reagents for extracting nucleic acids from a tissue section containing substantially degraded messenger RNA; and,
(b) an RNAse.

18. The kit according to claim 17 wherein the reagents for extracting nucleic acids from the tissue section containing substantially degraded messenger RNA comprise xylene and ethanol, and the tissue section is paraffin-embedded.

19. The kit according to claim 17 wherein the RNAse is RNAse I.

20. The kit according to claim 17 further comprising a DNAse.
